(19) United States
(12) Patent Application Publication
(10) Pub. No.: US 2005/0256393 A1
(43) Pub. Date: Nov. 17, 2005

(54) System and method for generating t1 and t2 maps

(76) Inventors: Sean Casey Louis Deoni, Ontario (CA); Brian Keith Rutt, Ontario (CA); Terence Malcolm Peters, Ontario (CA)

Correspondence Address: SIM & MCBURNEY, 330 UNIVERSITY AVENUE, 6TH FLOOR, TORONTO, ON, M5G 1R7, CA

(21) Appl. No.: 10/510,692

(22) PCT Filed: Apr. 8, 2003
(86) PCT No.: PCT/CA03/00497

§ 371 Date: Jun. 25, 2005

(30) Foreign Application Priority Data
Apr. 8, 2002 (US) 60370536

(51) Int. Cl. A61B 5/05

(52) U.S. Cl. 600/410; 600/407

(57) ABSTRACT

A method of generating three-dimensional T₁ and T₂ maps for steady state imaging involves acquiring a first set of spoiled gradient echo images with contrast dependent on T₁. A second set of fully refocused gradient echo images with contrast dependent upon T₁ and T₂ is then acquired. A T₁ map is generated from the first set of images and a T₂ map is generated from the second set of fully refocused gradient echo images and the T₁ map.

### TECHNICAL FIELD

The present invention relates generally-to magnetic resonance imaging and more specifically to a system and method for generating T₁ and T₂ maps. The present invention also relates to a system and method for determining pulse sequence flip angles and to a system and method for generating intensity corrected T₁ and T₂ weighted images.

### BACKGROUND ART

Magnetic resonance imaging or nuclear magnetic resonance (hereinafter referred to as “MRI”) is a well-known imaging technique. During MRI, a target, typically a human patient, is placed into the bore of an MRI machine and subjected to a uniform magnetic field produced by a polarizing magnet housed within the MRI machine. Radio frequency (RF) pulses, generated by an RF coil housed within the MRI machine in accordance with a particular localization method, are typically used to scan target tissue of the patient. MRI signals are radiated by excited nuclei in the target tissue in the intervals between consecutive RF pulses and are sensed by the RF coil. During MRI signal sensing, gradient magnetic fields are switched rapidly to alter the uniform magnetic field at localized areas thereby allowing spatial localization of MRI signals radiated by selected slices of the target tissue. The sensed MRI signals are in turn digitized and processed to reconstruct images of the target tissue slices using one of many known techniques.

When target tissue is subjected to a uniform polarizing magnetic field B₀, the individual magnetic moments of the spins in the tissue attempt to align with the polarizing magnetic field B₀, but precess about the polarizing magnetic field B₀ in random order at their characteristic Larmor frequency. The net magnetization vector lies along the direction of the polarizing magnetic field B₀ and is referred to as the equilibrium magnetization M₀. In this configuration, the Z component of the magnetization or longitudinal magnetization M_{z} is equal to the equilibrium magnetization M₀. If the target tissue is subjected to an excitation magnetic field B₁, which is in the x-y plane and which is near the Larmor frequency, the longitudinal magnetization M_{z} may be rotated, or “tipped” into the x-y plane to-produce a net transverse magnetic moment M_{xy}. When the excitation magnetic field B₁ is terminated, a signal is emitted by the excited spins that effects the magnitude of radiated MRI signals.

In particular, when the excitation magnetic field B₁ is terminated, the longitudinal magnetization M_{z} relaxes back to its equilibrium. The time constant that describes how the longitudinal magnetization M_{z} returns to its equilibrium value is commonly referred to as the spin lattice relaxation time T₁. T₁ characterizes the time required to reduce the difference between the longitudinal magnetization M_{z} and its equilibrium value M₀ to zero.

The net transverse magnetic moment M_{xy} also decreases back to its equilibrium when the excitation magnetic field B₁ is terminated. The time constant that describes how the transverse magnetic moment M_{xy} returns to its equilibrium value is commonly referred to as the spin-spin relaxation time T₂. T₂ characterizes the time required to reduce the transverse magnetic moment M_{xy} to zero. Both T₁ and T₂ are tissue specific and vary with concentration of different chemical substances in the tissue as well as with different microstructural features of the tissue. Variations of T₁ and/or T₂ from normal can also be indicative of disease or injury. As will be appreciated, measuring T₁ and T₂ at each point within an image to yield T₁ and T₂ maps can provide important diagnostic information.

Distinct from conventional clinical T₁ and T₂ weighted imaging techniques, it is often necessary to invoke imaging methods that permit calculation of Tand/or T₂ parameters explicitly. While pulse sequences used to produce standard multi-slice two-dimensional or three-dimensional T₁ and/or T₂ weighted images are very efficient (i.e. provide a high signal-to-noise (SNR) ratio per unit scan time), similar efficiencies have not so far been achieved when using pulse sequences designed to produce explicit maps of T₁ and/or T₂ in three dimensions.

Historically, various methods have been utilized to measure T₁ or T₂ relaxation times and thereby generate T₁ or T₂ maps. These methods. include, but are not restricted to, Inversion Recovery (IR), Saturation Recovery (SR) and Look-Locker (LL) for T₁ and Spin Echo (SE), fast Spin Echo (FSE), and SNAPSHOT-FLASH for T₂.

The main disadvantage of the aforementioned T₁ mapping techniques is their dependence upon long repetition periods (TR) between consecutive RF pulses in the pulse sequence. These inter-excitation repetition periods TR should be up to 5 times the length of T₁. Depending on the target tissue of interest, this corresponds to repetition periods TR between 3.5 to 20 seconds. The end result is scan times of approximately 200 hours in order to produce a 256×256×100 voxel T₁ map, where 8 echo times are acquired per voxel.

LL makes use of an inversion pulse followed by a train of low angle RF pulses generated at regular intervals, with the transverse magnetization being destroyed prior to each RF pulse in the pulse train. In this way, the magnetization is driven back to equilibrium with an apparent recovery time constant T₁* which is related to T₁ in a known way. The imaging time for a single 256×256 pixel slice with precision comparable to IR is about 10 minutes, which is a significant time savings when compared to the IR and SR techniques. However, serial collection of large numbers of slices brings the LL scan time back to clinically unrealistic values.

Similar to the above-described T₁ mapping techniques, the primary disadvantage of the aforementioned T₂ mapping techniques is their dependence upon long repetition periods TR between consecutive RF pulses in the pulse sequence, which are necessary in order to eliminate T₁ effects. As a result, approximately 200 hours of scanning is required in order to produce a 256×256×100 voxel T₂ map using the SE method, where 8 echo times are acquired per voxel. This scanning time can be reduced by up to two orders of magnitude by using slice interleaving and FSE strategies. However, the use of slice selective refocusing image pulses often induces errors in the T₂ measurement.

SNAPSHOT-FLASH is an alternative approach for T₂ measurement which, when combined with a prior T₁ measurement technique, reduces the required repetition period TR between consecutive RF pulses in the pulse sequence significantly. Unfortunately, this method still requires at least 1.5 hours of scanning to produce a 256×256×100 voxel T₂ map and is computationally expensive since non-linear fitting is required to extract the T₂ map from the MRI signals.

As will be appreciated, in order to deliver effective clinical T₁ and/or T₂ mapping, a decrease in acquisition and computation times is required while maintaining high signal-to-noise ratios and avoiding special hardware requirements.

It is therefore an object of the present invention to provide a novel system and method for generating T₁ and T₂ maps. It is also an object of the present invention to provide a novel system and method for determining pulse sequence flip angles and a system and method for generating intensity corrected T₁ and T₂ weighted images.

### DISCLOSURE OF THE INVENTION

The present invention relates generally to magnetic resonance imaging (MRI) and includes a method of generating T₁ and T₂ maps in a time comparable to clinical T₁ and/or T₂ weighted images, which can be performed using clinical MRI scanners, provides high signal-to-noise and has rapid, “real-time” post-processing.

In accordance with one aspect of the present invention there is provided a method of generating a T₂ map comprising the steps of:
acquiring a set of fully refocused gradient echo images with contrast dependent upon both T₁ and T₂; and
generating said T₂ map using the set of fully refocused gradient echo images and T₁ information.

In accordance with another aspect of the present-invention there is provided a method for generating three-dimensional T₁ and T₂ maps with steady state imaging comprising the steps of:
acquiring a first set of spoiled gradient echo (SPGR) images with contrast dependant primarily on T₁;
acquiring a second set of fully refocused gradient echo (SSFP) images with contrast dependent upon both T₁ and T₂;
generating a T₁ map from the first set of images; and
generating a T₂ map from using the second set of images and the T₁ map.

In accordance with another aspect of the present invention there is provided a method of generating a T₂ map comprising the steps of:
acquiring a set of images with contrast dependent on T₁ and T₂ at different flip angles, said images being generated from signals defined by a linearizable equation; and
generating said T₂ map from said signals and from T₁ information using a linearized form of said equation.

In accordance with yet another aspect of the present invention there is provided a method of generating an intensity corrected T₁-weighted image comprising the steps of:
generating an explicit T₁ map; and
substituting values of the T₁ map into an MR signal equation to generate a corrected T₁-weighted image.

In accordance with still yet another aspect of the present invention there is provided a method of generating an intensity corrected T₂-weighted image comprising the steps of:
generating an explicit T₂ map; and
substituting values of the T₂ map into an MR signal equation to generate a corrected T₂-weighted imaged.

Analytical expressions are also disclosed which allow for calculation of the RF flip angles to be used in both sequences to enhance the precision of the T₁ and T₂ maps.

### BRIEF DESCRIPTION OF THE DRAWINGS

Embodiments of the present invention will now be described more fully with reference to the accompanying drawings in which:

FIG. 1 is a schematic block diagram of an MRI system;

FIG. 2 is a block diagram of a method of generating T₁ and T₂ maps in accordance with the present invention;

FIG. 3 is a timing diagram of a simplified two-dimensional MRI pulse sequence used during the method of FIG. 2 for T₁ mapping in the steady state;

FIG. 4 is a timing diagram of a simplified two-dimensional MRI pulse sequence used during the method of FIG. 2 for T₂ mapping;

FIG. 5 is a signal intensity versus flip angle curve for a spoiled gradient echo pulse sequence showing determination of pulse sequence flip angles;

FIG. 6 is a signal intensity versus flip angle curve for a fully refocused gradient echo pulse sequence showing determination of pulse sequence flip angles;

FIG. 7 is a block diagram of the steps used to generate an intensity corrected T₁ weighted image in accordance with the present invention;

FIG. 8 is a 256×256×100 voxel T₁ map of a tissue slice of the brain of a normal volunteer, generated in accordance with the present invention;

FIGS. 9a to 9c are T₁ maps of approximately the same tissue slice acquired using IR, SR and the method of FIG. 2, respectively;

FIG. 10 is a 256×256×100 voxel T₂ map of a tissue slice of the brain of a normal volunteer, generated in accordance with the present invention; and

FIGS. 11a to 11c are T₂ maps of approximately the same tissue slice acquired using SE, FSE and the method of FIG. 2, respectively.

### BEST MODE FOR CARRYING OUT THE INVENTION

In one aspect, the present invention relates generally to a system and method of generating a T₂ map in a time comparable to that required during generation of a T₂weighted image. During the T₂ map generation, a set of fully refocused images of a target with contrast dependent upon both T₁ and T₂ is acquired. From the set of fully refocused images and previously acquired T₁ information, the T₂ map is generated. Preferably, the T₁ information is a T₁ map generated from a set of spoiled images with contrast dependent primarily on T₁. In order to generate the T₁ and T₂ maps in clinically realistic time frames, the signals used to generate the T₁ and T₂ maps are expressed in linear form and linear techniques are used to determine the values for the T₁ and T₂ maps in a computationally efficient manner.

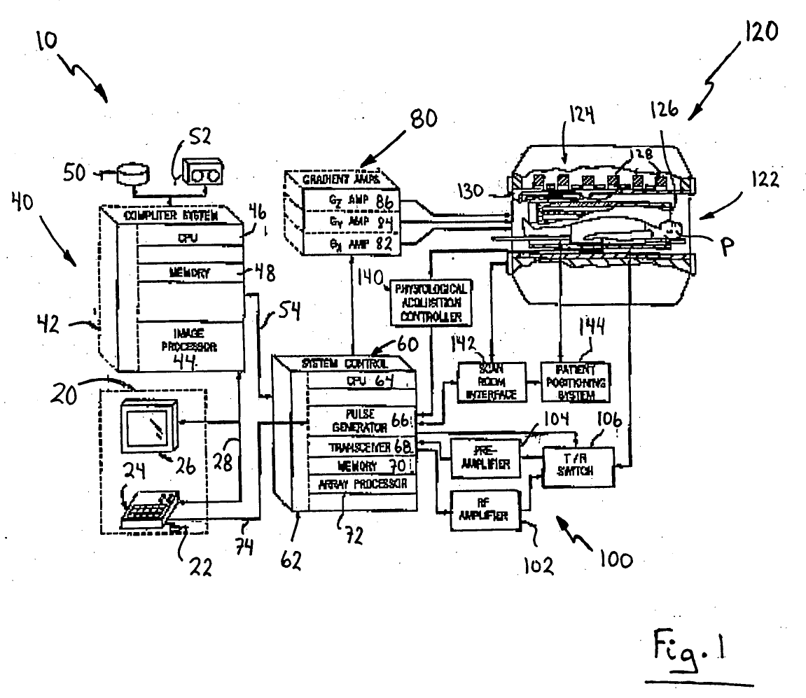

In the preferred embodiment, a three-dimensional T₂ map is generated from a set of fully refocused gradient echo (SSFP) images acquired in response to a three-dimensional pulse sequence. The T₁ map is preferably generated from a set of spoiled gradient echo (SPGR) images in response to a three-dimensional pulse sequence. Further specifics of the present invention will now be described with particular reference to FIGS. 1 to 11c.

Turning now to FIG. 1, an MRI system is shown and is generally identified by reference numeral 10. The MRI system 10 includes an operator console 20, a computer system 40, a system controller 60, a gradient amplifier system 80, an RF coil control circuit 100 and an MRI machine 120. The operator console 20 allows an operator to control the MRI system 10 including the production and display of images. The computer system 40 is responsive to commands generated by the operator console 120 and generates images for display. The system controller 60 communicates with the operator console 20, the computer system 40, the gradient amplifier system 80 and the RF coil control circuit 100 and orchestrates the acquisition of images in response to commands generated by the operator console 20. The MRI machine 120 communicates with the gradient amplifier system 80 and the RF coil control circuit 100.

The operator console 20 includes an input device 22, a control panel 24 coupled to the input device 22, and a display 26. The input device 22 can include a mouse, joystick, keyboard, trackball, touch screen, light wand, voice control, or similar such device, and may be used for interactive geometry prescription. The operator console 20 communicates with the computer system 40 over a data communications link 28 thereby to enable an operator to control the production and presentation of images on the display 26.

The computer system 40 includes a number of modules, which communicate with each other through a backplane 42. As can be seen, the modules of computer system 40 include an image processor module 44, a CPU module 46, and a memory buffer 48, known in the art as a frame buffer for storing image data arrays. The computer system 40 is linked to a disk storage 50 and a tape drive 52 for storage of image data and programs. The computer system 40 communicates with the system controller 60 over a high-speed serial data communications link 54.

The system controller 60 also includes a number of modules, which communicate with each other through a backplane 62. The modules of system controller 60 include a CP module 64, a pulse generator module 66, a transceiver module 68, a memory module 70 and an array processor module 72. The pulse generator module 66 communicates with the operator console 20 over a serial data communications link 74.

The gradient amplifier system 80 includes Gₓ, G_{y} and G_{z} gradient amplifiers 82 to 86 respectively. The gradient amplifiers 82 to 86 receive input gradient pulse data from the system controller 60 and generate output gradient pulses that are conveyed to the MRI machine 120.

The RF coil control circuit 100 includes an output RF amplifier 102, an input RF preamplifier 104 and a transmit/receive (T/R) switch 106. The output RF amplifier 102 and input RF preamplifier 104 communicate with the transceiver module 68 of the system controller 60. The T/R switch 106 is coupled to the MRI machine 120 and to the RF amplifier 102 and RF preamplifier 104.

The MRI machine 120 includes a bore 122 to receive a patient P. A magnet assembly 124 and a whole-body RF coil 126 are disposed within the MRI machine 120. The magnet assembly 124 includes a polarizing magnet 128 to generate a uniform magnetic field and gradient coils 130 that are responsive to the output gradient signals generated by the gradient amplifiers 82 to 86. Each gradient coil 130 is associated with a respective one of the gradient amplifiers. The whole-body RF coil 126 is coupled to the T/R switch 106.

In addition to the above components, the MRI system 10 includes a physiological acquisition controller 140, a scan room interface circuit 142 and a patient positioning system 144. The physiological acquisition controller 140 is coupled to the pulse generator module 66 and to the MRI machine 120. The scan room interface circuit 142 is coupled to the pulse generator module 66, the patient positioning system 144 and the MRI machine 120. The patient positioning system 144 is also coupled to the MRI machine 120. The physiological acquisition controller 140 receives signals from a number of different sensors connected to the patient P, such as ECG signals from electrodes attached to the patient, and conveys the signals to the pulse generator module 66. The scan room interface circuit 142 receives input from various sensors associated with the condition of the patient and the magnet assembly 124 and conveys the signals to the pulse generator module 66. The patient positioning system 144 receives commands from the scan room interface circuit 142 and in response moves the patient P within the MRI machine 120 to the desired location for the scan.

The general operation of the MRI system 10 will firstly be described for ease of understanding. During imaging, the patient P within the MRI machine 120 is subjected to a uniform magnetic field produced by the polarizing magnet 128. RF pulses are then generated by the whole-body RF coil 126 in a particular sequence and are used to scan target tissue of the patient. MRI signals radiated by excited nuclei in the target tissue in the intervals between consecutive RF pulses are sensed by the whole-body RF coil 126. During this MRI signal sensing, the uniform magnetic field is altered by the gradient coils 130 in response to received output gradient data thereby to position encode acquired MRI signals.

The sequence of RF pulses used to scan the patient P is generated by the whole-body RF coil 126 in response to pulse sequence data received from the pulse generator module 66 of the system controller 60 via the transceiver module 68 and RF coil control circuit 100. The pulse sequence data determines the timing, strength and shape of the RF pulses in the pulse sequence as well as the MRI signal acquisition window. The RF sequence data is generated by the pulse generator module 66 in response to scan commands received from the operator console 20 via the data communications link 74.

When an RF pulse is to be applied to the target tissue, the RF coil control circuit 100 is conditioned to a transmit mode by the pulse generator module 66. In the transmit mode, the T/R switch 106 couples the output RF amplifier 102 to the whole-body RF coil 126. RF pulse data generated by the pulse generator module 66 is converted into an RF pulse via the transceiver module 68 and RF amplifier 102 and applied to the whole-body RF coil 126.

The pulse generator module 66 also generates gradient data in response to the scan commands received from the operator console 20 via the data communications line 74 and conveys the gradient data to the gradient amplifier system 80. The gradient data determines the timing and shape of the output gradient pulses generated by the gradient amplifiers 82 to 86 that are applied to the gradient coils 130 during scanning.

During MRI signal sensing in the MRI signal acquisition window, the pulse generator module 66 conditions the RF coil control circuit 100 to a receive mode. In the receive mode, the T/R switch 106 couples the input RF preamplifier 104 to the whole-body RF coil 126.

The MRI signals radiated by excited nuclei in the target tissue are sensed by the whole-body RF coil 56 and conveyed to the transceiver module 68 via the T/R switch 106 and input RF preamplifier 104. The amplified MRI signals are in turn demodulated, filtered and digitized by the transceiver module 68 and then transferred to the memory module 70.

After a scan of the target tissue is completed, an array of raw k-space data is stored in the memory module 70. The array processor 72 Fourier transforms the raw k-space data into an array of image data that is conveyed through the serial data communication link 54 to the computer system 20 where it is stored in the disk memory 50. In response to commands received from the operator console 20, the image data may be archived on the tape drive 52, or it may be further processed by the image processor 44 and conveyed to the operator console 20 for presentation on the display 26.

In accordance with the present invention, the MRI system 10 is operated in a manner to generate explicit T₁ and T₂ maps from the sensed MRI signals. This is achieved by conditioning the MRI system to acquire specific types of images of the target tissue at different flip angles (α) while maintaining the repetition period TR between consecutive RF pulses in the pulse sequence constant and to process the image data to generate the explicit T₁ and T₂ maps.

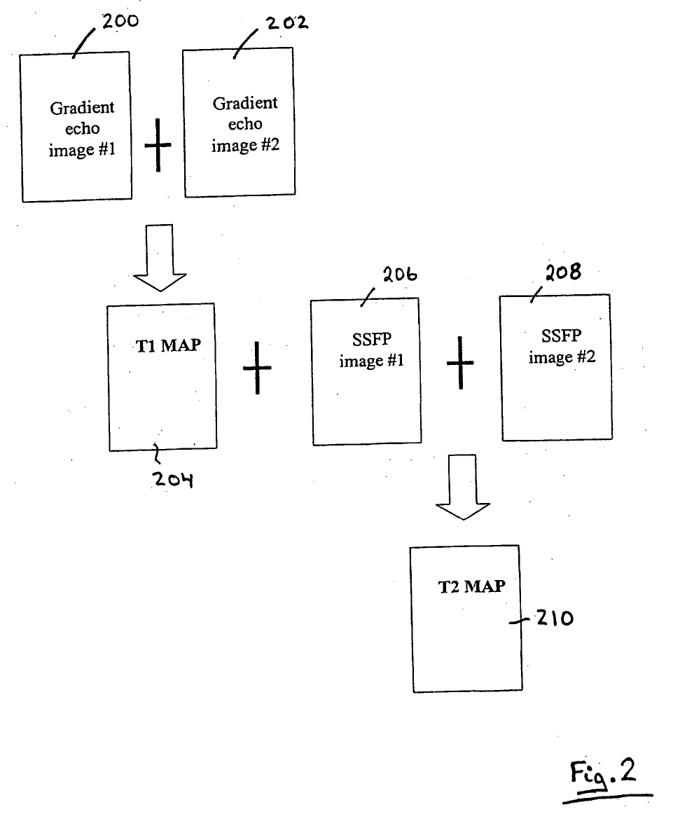

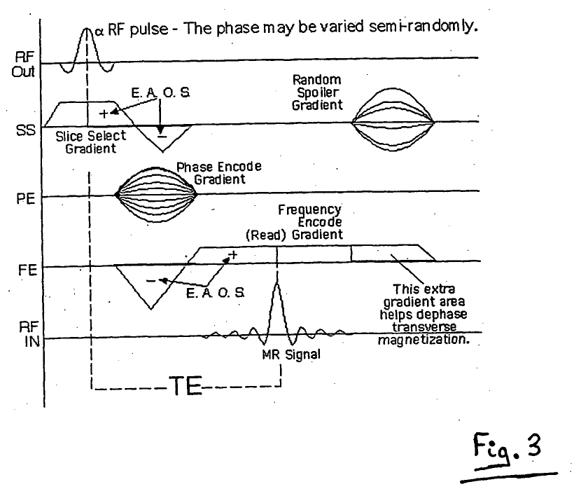

Turning now to FIG. 2, the method by which the MRI system 10 is operated to generate the explicit T₁ and T₂ maps is shown. Initially, the MRI system 10 is conditioned to acquire at least two spoiled gradient echo (SPGR) images each at a different flip angle (α). FIG. 3 shows simplified RF and gradient pulses used by the MRI system to acquire two-dimensional spoiled gradient echo images. In the preferred embodiment, a three-dimensional pulse sequence is used to acquire the SPGR images 200 and 202. The repetition period TR between consecutive RF pulses in the pulse sequence is held constant. The acquired SPGR images are then processed in a fast and efficient manner to yield an explicit T₁ map 204 as will be described.

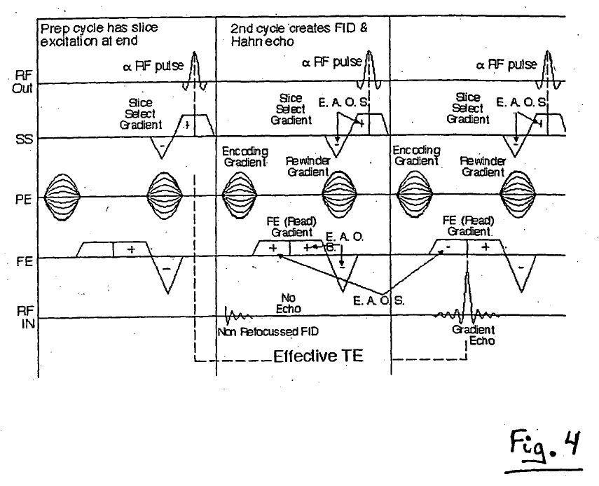

With the explicit T₁ map 204 generated, the MRI system 10 is then conditioned to acquire at least two fully refocused gradient echo (SSFP) images 206 and 208, each at a different flip angle (α). FIG. 4 shows simplified RF and gradient pulses used by the MRI system 10 to acquire two-dimensional SSFP images. In the preferred embodiment, a three-dimensional pulse sequence is used to acquire the SSFP images 206 and 208. The repetition period TR between consecutive RF pulses in the pulse sequence is held constant. The acquired SSFP images 206 and 208 together with the previously generated explicit T₁ map 204 are then processed in a fast and efficient manner-to yield an explicit T₂ map 210 as will be described. As a result, the MRI system 10 enables explicit T₁ and T₂ maps 204 and 210 to be generated in clinically realistic times, a result that heretofore has been unavailable. Further specifics of the manner by which the SPGR and SSFP images are processed to generate the explicit T₁ and T₂ maps will now be described.

The MRI signals S used to generate each of the acquired SPGR images are given by:

$$S = \frac{{M}_{o} ( 1 - {E}_{1} ) sin ( α )}{1 - {E}_{1} cos ( α )} \\ where \text{:} \\ {E}_{1} = exp ( \frac{- TR}{{T}_{1}} ) \qquad ( 1 )$$

and where:
M₀ is a term proportional to the equilibrium magnetization;
TR is the repetition period; and
α is the flip angle.

Equation (1) can be rewritten in a linear form (Y=mX+b) as:

$$\frac{S}{sin ( α )} = {E}_{1} \frac{S}{tan ( α )} + {M}_{o} ( 1 - {E}_{1} ) \qquad ( 2 )$$

By holding the repetition period TR constant and varying the flip angle (α), an MRI signal vs. flip angle curve is provided that is easily linearized according to equation (2). Recasting the raw signal values as:
S/sin(α) vs S/tan(α)
allows for the determination of T₁ and Mₒ by determining the slope of the linear relationship through linear regression as:

$$\begin{matrix}{T}_{1} = \frac{- TR}{ln ( m )} & ( 3 ) \\ {M}_{o} = \frac{b}{1 - m} & ( 4 )\end{matrix}$$

Performing the above-described analysis on the acquired spoiled gradient echo image data yields the T₁ map 204.

The flip angles (α) that yield that best results are determined through the behavior of the points along the regression line in the linearized space, which is termed “regression-space”. If the data points all suffer the same uncertainty the further the two points are separated along the regression line, the better the estimate of slope. This separation along the abscissa can be defined as the normalized dynamic range (DR):

$$DR = \frac{{S}_{α2}}{{M}_{o} sin ( {α}_{2} )} - \frac{{S}_{α1}}{{M}_{o} sin ( {α}_{1} )} \qquad ( 5 )$$

where Sα1 and Sα2 are the MRI signals associated with flip angles α1 and α2 respectively. In the above case, the points do not all suffer the same uncertainty. Rather, the uncertainty is a function of the point location and generally increases as the point moves away from the mid-point. Since the mid-point is defined by the Ernst signal (SαE), the uncertainty can be related to the fractional signal FS of Sα1 and Sα2 to SαE:

$$FS = \frac{S α 1 + S α2}{2 S α E} \qquad ( 6 )$$

Thus, there is a trade-off between DR and FS and the best T₁ map results where the product of FS and DR is maximized.

This generally occurs when α1=α2 and so equation (6) simplifies to:

$$f = \frac{S α1}{S α E} = \frac{S α 2}{S α E} \qquad ( 7 )$$

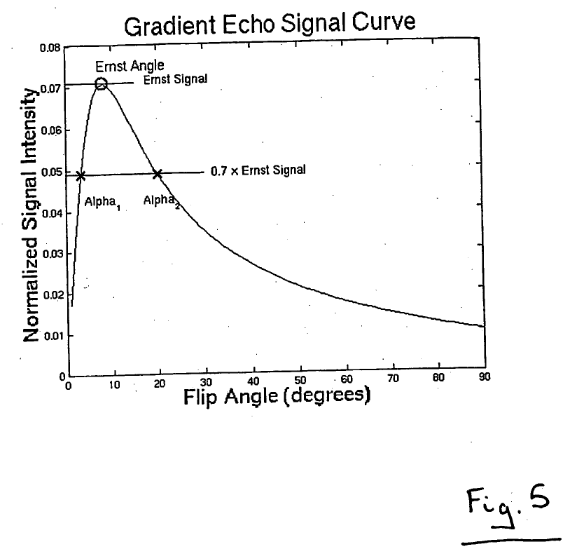

Further, maximum precision is obtained when f=0.7. Thus, the flip angles (α) that have been found to yield the best T₁ map are those that provide 70% of the Ernst signal as illustrated in FIG. 5. These preferred flip angles can be computed as follows:

$$α = {cos}^{- 1} ( \frac{{f}^{2} {E}_{1} \pm ( 1 - {E}_{1}^{2} ) \sqrt{1 - {f}^{2}}}{1 - {E}_{1}^{2} ( 1 - {f}^{2} )} ) \qquad ( 8 )$$

The MRI signals S used to generate each of the SSFP images are given by:

$$S = \frac{{M}_{o} ( 1 - {E}_{1} ) sin ( α )}{1 - {E}_{1} {E}_{2} - ( {E}_{1} - {E}_{2} ) cos ( α )} \\ \text{where:} \\ {E}_{1} = exp ( \frac{- TR}{{T}_{1}} ) ; and {E}_{2} = exp ( \frac{- TR}{{T}_{2}} ) \qquad ( 9 )$$

and where:
M₀ is a term proportional to the equilibrium magnetization;
TR is the repetition period; and
α is the flip angle.

Equation (9) can also be rewritten in linear form (Y=mX+b) using the following linearization:

$$\frac{S}{sin ( α )} = \frac{S}{tan ( α )} \times \frac{{E}_{1} - {E}_{2}}{1 - {E}_{1} {E}_{2}} + \frac{{M}_{o} ( 1 - {E}_{1} )}{1 - {E}_{1} {E}_{2}} \qquad ( 10 )$$

By holding the repetition period TR constant and varying the flip angle (α), an MRI signal vs. flip angle curve is provided that is easily linearized according to equation (10). Thus, plotting:
S/sin(α) vs S/tan(α)
allows for the determination of T₂ and Mₒ by determining the slope of the linear relationship through linear regression as:

$$\begin{matrix}{T}_{2} = \frac{- TR}{ln ( \frac{m - {E}_{1}}{{mE}_{1} - 1} )} & ( 11 ) \\ {M}_{o} = \frac{b ( {E}_{1} {E}_{2} - 1 )}{1 - {E}_{1}} & ( 12 )\end{matrix}$$

Thus, with prior knowledge of T₁ and therefore E₁, performing the above-described analysis on the SSFP image data yields the T₂ map 210.

As in the T₁ case, the best flip angles (α) for the SSFP images are determined through maximization of the DR×FS product. Once again, FS can be simplified to:

$$f = \frac{S α1}{S α E} = \frac{S α2}{S α E} \qquad ( 13 )$$

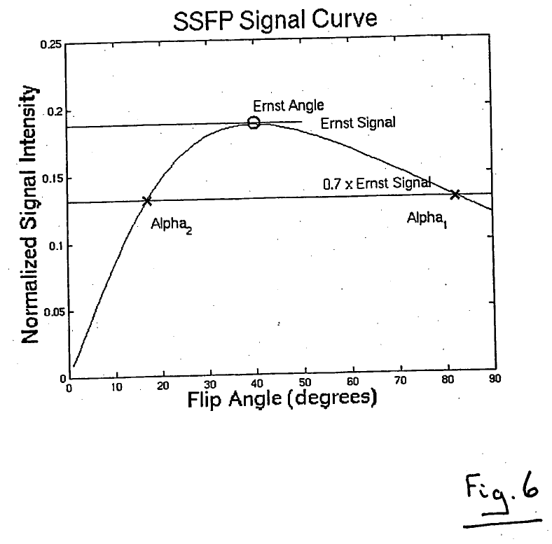

Maximum T₂ precision is achieved when f=0.7 as shown in FIG. 6. The two flip angles are therefore found by the solution of the quadratic equation in cos(α):
A cos²(α)+B cos(α)+C=0
where:
A=2E₁E₂+2E₁E₂ Ψ−2E₁E₂(E₁−E₂)Ψ+E₁²E₂²+(E₁−E₂)²Ψ²−f²(1−Ψ)(E₁−E₂)²
B=2f²(1−Ψ)(E₁−E₂ )−2f²(1−Ψ)E₁E₂(E₁−E₂)
C=1−2E₁E₂−2(E₁−E₂)Ψ+2E₁E₂(E₁−E₂)Ψ+E₁²E₂²+(E₁−E₂)²Ψ²−f²(1−Ψ)+2 f²(1−Ψ)E₁E₂−f²(1−Ψ)E₁²E₂²

$$Ψ = \frac{{E}_{1} - {E}_{2}}{1 - {E}_{1} {E}_{2}}$$

Due to the ability to linearize both signal equations (1) and (4), image acquisition time is minimized, as only two images are required to calculate each relaxation time map. Additionally, linear regression is far less computationally expensive than iterative non-linear regression, allowing the T₁ and T₂ maps to be computed in near “real-time” compared to the minutes or hours presently required by the conventional techniques.

With a practical repetition period TR of less than 6 ms, all required 256×256×100 voxel images can be acquired in 20 minutes, or five minutes for each image.

Substituting images containing contrast based on T₁ and T₂* for the fully refocused gradient echo images described above allows one to solve for T₂* either additionally or instead of T₂.

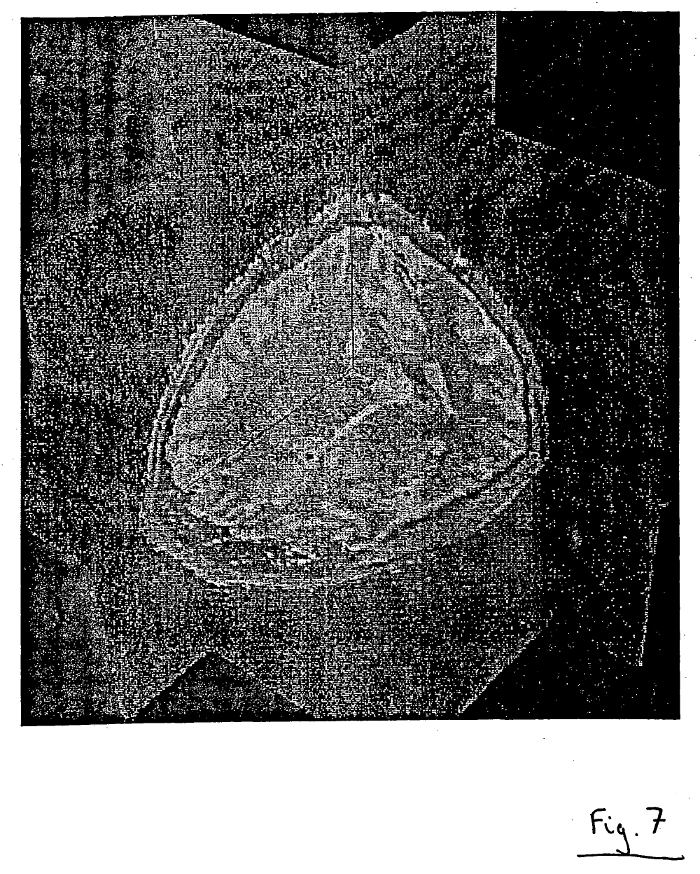

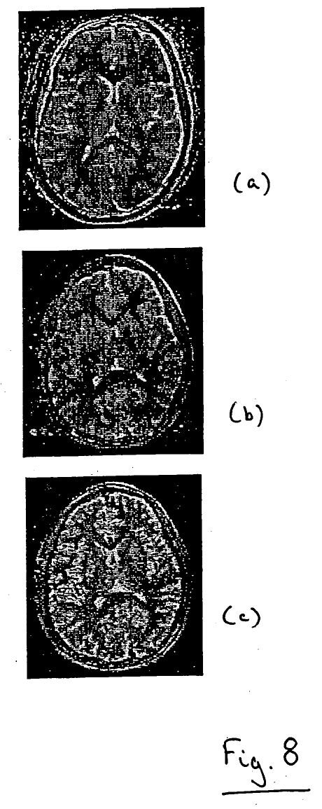

FIG. 7 shows a 256×256×100 voxel T₁ of a tissue slice of the brain of a normal volunteer generated in accordance with the present invention. For comparison, FIGS. 8a to 8c show T₁ maps of approximately the same tissue slice acquired using IR, SR and the present method.

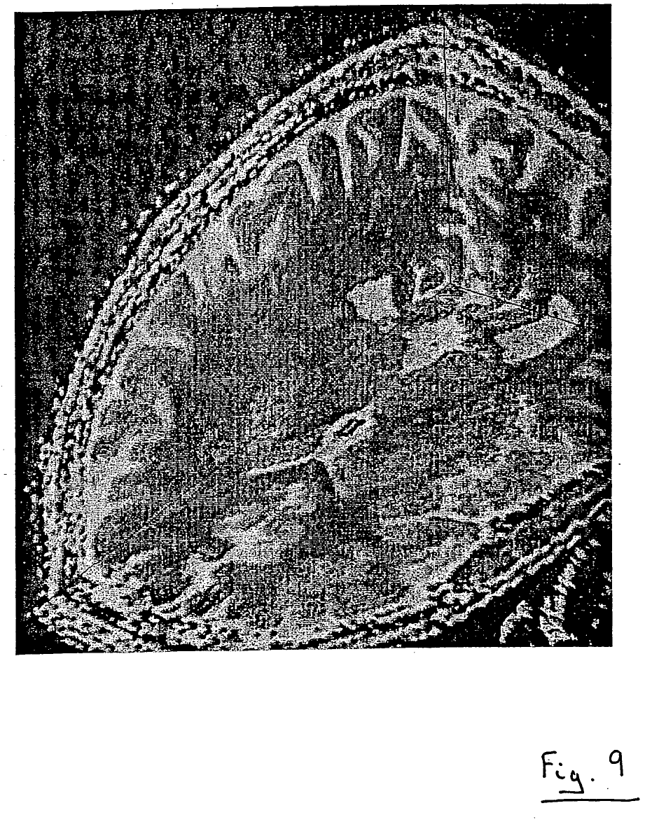

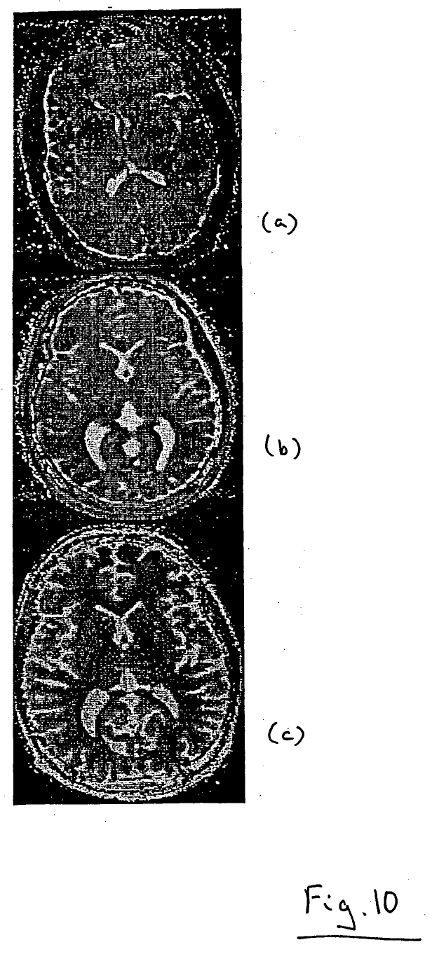

FIG. 9 is a 256×256×100 voxel T₂ map of a tissue slice of the brain of a normal volunteer generated in accordance with the present method. For contrast, FIGS. 10a to 10c show T₂ maps of approximately the same tissue slice acquired using SE, FSE and the present method.

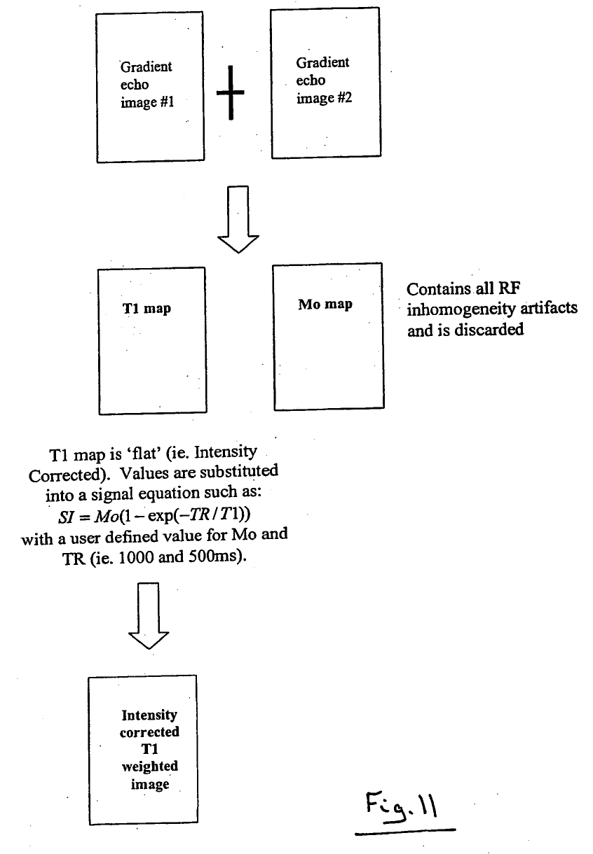

Referring now to FIG. 11 shows how RF inhomogeneity artifacts, or abnormal signal intensity variations across an MR image can be corrected using the T₁ mapping component of the present invention. Following the acquisition of the SPGR images containing these artifacts, the T₁ and Mₒ values may be calculated as previously described. Since T₁ is a property of the tissue itself, it is unaltered by these signal variations. As a result the T₁ map calculated from the SPGR images will be the same as if it were calculated from ideal artifact free images. Following the calculation of the T₁ map, the T₁ values may be substituted into any MR signal equation (i.e. IR, SR, SPGR) along with the other necessary constants (i.e. TR, TE, TI, α) to generate a new intensity corrected T₁-weighted image.

As will be appreciated by those of skill in the art, an MRI image can also be corrected using the T₂ mapping component of the present invention. In this case, following the acquisition of the SSFP images containing artifacts, the T₂ values of the T₂ map may be substituted into any MR signal equation along with the other necessary constants to generate a new intensity corrected T₂-weighted image.

In the preferred embodiment, the SPGR and SSFP images are derived using three-dimensional pulse sequences. Those of skill in the art will however appreciate that two-dimensional or three-dimensional pulse sequences may be used to acquire the images. For T₂ mapping, any pulse sequence that is fully refocused can be used and for T₁ mapping, any pulse sequence that is spoiled can be used allowing arbitrary k-space trajectories to be used instead of Cartesian k-space trajectories.

The preferred method of generating T₁ and T₂ maps has been described with reference to acquiring two SPGR images and two SSFP images at particular flip angles. It will be appreciated that in accordance with the present invention, any number of spoiled images and fully refocused images at different flip angles can be acquired and used to generate the T₁ and T₂ maps. Although T₁ and T₂ maps will generally be generated using two or more spoiled images and two or more fully refocused images at different flip angles, T₁ and T₂ maps can be generated using spoiled and fully refocused images at a single flip angle if other information is available.

For example, in the case of T₁ mapping, if an M₀ map is available from a prior M₀ mapping sequence, then a T₁ map may be computed from the M₀ map and the spoiled image. In the case of T₂ mapping, if M₀ and T₁ maps are available from prior acquisitions, then a T₂ map can be computed from the M₀ and T₁ maps and the fully refocused image.

Although the present method is described through examples that involve processing the spoiled and fully refocused images, those of skill in the art will appreciate that the raw spoiled and fully refocused k-space data may be processed to generate the T₁ and T₂ maps.

During generation of the T₂ map, in the preferred embodiment the SSFP images and a T₁ map derived from the spoiled gradient echo images are processed. Those of skill in the art will however appreciate that T₁ information derived using other T₁ mapping methods may of course be used.

Although preferred embodiments of the present invention have been described, those of skill in the art will appreciate that variations and modifications may be made without departing from the spirit and scope thereof as defined by the appended claims.

### REFERENCES

K A Christensen, D M Grand, E M Schulman, C Walling, “Optimal Determination of Relaxation T₁ Times of Fourier Transform Nuclear Magnetic Resonance. Determination of Spin-Lattice Relaxation Times of Chemically Polarized Species”, Journal of Physical Chemistry, 78, pp., 1971-1977 (1974)
J Homer, M S Beevers, “Driven Equilibrium Single Pulse Observation of T₁ relaxation, A Re-evaluation of a Rapid ‘New’ Method for Determining NMR Spin-Lattice Relaxation Times”, Journal of Magnetic Resonance, 63, pp., 287-297 (1985)
S C L Deoni, B K Rutt, T M Peters, “Rapid Combined T₁ and T₂ Mapping using Gradient Recalled Acquisition in the Steady State. Magnetic Resonance in Medicine 49(3): 515-26 (2003)

1. A method of generating a T₂ map comprising the steps of:
acquiring a set of fully refocused gradient echo images with contrast dependent upon both T₁ and T₂; and
generating said T₂ map using the set of fully refocused gradient echo images and T₁ information.

2. The method of claim 1 wherein said T₁ information is a T₁ map.

3. The method of claim 2 wherein said set of fully refocused gradient echo images include T₁ and T₂ weighted contrast.

4. The method of claim 3 wherein said set of fully refocused images includes at least two fully refocused gradient echo (SSFP) images.

5. The method of claim 4 wherein said SSFP images are acquired using a three-dimensional pulse sequence.

6. The method of claim 5 wherein each SSFP image is acquired at a different flip angle and wherein the repetition time between consecutive pulses in said pulse sequence is held constant.

7. The method of claim 6 wherein said T₂ map is generated from said SSFP images and T₁ map using a linearization technique.

8. The method of claim 7 wherein the equations defining the signals used to form said SSFP images are linearized and wherein said T₂ map is generated from said signals and T₁ map using linear regression.

9. The method of claim 8 wherein the equation defining the signals used to form said SSFP images is:
$$S = \frac{{M}_{o} ( 1 - {E}_{1} ) sin ( α )}{1 - {E}_{1} {E}_{2} - ( {E}_{1} - {E}_{2} ) cos ( α )}$$
$$where \text{:}$$
$${E}_{1} = exp ( \frac{- TR}{{T}_{1}} ) ; and$$
$${E}_{2} = exp ( \frac{- TR}{{T}_{2}} )$$
and where:
M₀ is a term proportional to the equilibrium magnetization;
TR is the repetition period; and
α is the flip angle.
and wherein said T₂ map is generated by applying linear regression to the following equations to determine the slope of linearized data:
$$\begin{matrix}{T}_{2} = \frac{- TR}{ln ( \frac{m - E}{{mE}_{1} - 1} )} & ( 11 ) \\ {M}_{o} = \frac{b ( {E}_{1} {E}_{2} - 1 )}{1 - {E}_{1}} & ( 12 )\end{matrix}$$

10. The method of any one of claims 1 to 9 wherein said T₁ information is calculated from a set of spoiled gradient echo images with contrast dependant primarily on T₁.

11. The method of claim 10 wherein said set of spoiled gradient echo images includes at least two spoiled gradient-echo (SPGR) images.

12. The method of claim 11 wherein said SPGR images are acquired using a three-dimensional pulse sequence.

13. The method of claims 12 wherein each SPGR image is acquired at a different flip angle and wherein the repetition time between consecutive. pulses in said pulse sequence is held constant.

14. The method of claim 13 wherein said T₁ map is generated from said SPGR images using a linearization technique.

15. The method of claim 14 wherein the equation defining the signals used to form said spoiled gradient echo images are linearized and wherein said T₁ map is generated from said signals using linear regression.

16. The method of claim 15 wherein the equation defining the signal used to form said SPGR images is:
$$S = \frac{{M}_{o} ( 1 - {E}_{1} ) sin ( α )}{1 - {E}_{1} cos ( α )}$$
and wherein said T₁ map is generated by applying linear regression to the following equations to determine the slope of linearized data:
$${T}_{1} = \frac{- TR}{ln ( m )}$$
$${M}_{o} = \frac{b}{1 - m}$$

17. A method for generating three-dimensional T₁ and T₂ maps with steady state imaging comprising the steps of:
acquiring a first set of spoiled gradient echo (SPGR) images with contrast dependant primarily on T₁;
acquiring a second set of fully refocused gradient echo (SSFP) images with contrast dependent upon both T₁ and T₂;
generating a T₁ map from the first set of images; and
generating a T₂ map from using the second set of images and the T₁ map.

18. The method of claim 17 wherein said T₁ map and T₂ map are generated using computationally efficient linearization techniques.

19. The method of claim 18 wherein the equations defining the signals used to form said SSFP images are linearized and wherein said T₂ map is generated from said signals and T₁ map using linear regression.

20. The method of claim 19 wherein the equation defining the signals used to form said SPGR images are linearized and wherein said T₁ map is generated from said signals using linear regression.

21. A method of generating a T₂ map comprising the steps of:
acquiring a set of images with contrast dependent on T₁ and T₂ at different flip angles, said images being generated from signals defined by a linearizable equation; and
generating said T₂ map from said signals and from T₁ information using a linearized form of said equation.

22. The method of claim 21 wherein said T₁ information is a T₁ map.

23. The method of claim 22 wherein said set of images include T₁ and T₂ weighted contrast.

24. The method of claim 23 wherein said set of images includes two or more images.

25. The method of claim 24 wherein said T₂ map is generated using linear regression.

26. A method of generating a T₂ map comprising the steps of:
acquiring a fully refocused image with contrast dependent on both T₁ and T₂; and
generating said T₂ map using the fully refocused image and previously acquired M₀ and T₁ maps.

27. The method of claim 26 wherein said fully refocused image is a fully refocused gradient echo image.

28. The method of claim 27 wherein said T₁ map is computed from the M₀ map and a spoiled image.

29. The method of claim 28 wherein the spoiled image is a spoiled gradient echo image.

30. A method of generating an intensity corrected T₁-weighted image comprising the steps of:
generating an explicit T₁ map; and
substituting values of the T₁ map into an MR signal equation to generate a corrected T₁-weighted image.

31. The method of claim 30 wherein the MR signal equation is one of an IR, SR or SPGR signal equation.

32. The method of claim 31 wherein the explicit T₁ map is generated using a set of spoiled gradient echo images.

33. A method of generating an intensity corrected T₂-weighted image comprising the steps of:
generating an explicit T₂ map; and
substituting values of the T₂ map into an MR signal equation to generate a corrected T₂-weighted imaged.

34. The method of claim 33 wherein the explicit T₂ map is generated using a set of fully refocused gradient echo images.
